(19) United States
(12) Patent Application Publication
(10) Pub. No.: US 2010/0297037 A1
(43) Pub. Date: Nov. 25, 2010

(54) MATERIAL COMPOSITION FOR ORAL HYGIENE PRODUCT

(76) Inventor: Klaus-Uwe Busch, Osterreich (AT)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 12/680,399

(22) PCT Filed: Oct. 2, 2008
(86) PCT No.: PCT/EP08/08386

§ 371 Date: Jul. 29, 2010

(30) Foreign Application Priority Data
Oct. 2, 2007 (DE) 10 2007 047 237.6

(51) Int. Cl. A61K 9/68; A61K 36/9066; A61K 8/49; A61P 1/02; A61P 29/00; A61K 31/4985

(52) U.S. Cl. 424/48; 424/756; 424/55; 514/249

(57) ABSTRACT

The present invention relates to a material composition, particularly for an oral hygiene product suitable for combating caries and also for combating parodontitis and gingivitis. Said material composition or oral hygiene product is characterized in that it comprises an extract of Curcuma xanthorrhiza or xanthorrhizol and folic acid.

### FIELD OF THE INVENTION

The present invention relates to a material composition, in particular for an oral hygiene product as e.g. toothpastes, oral rinses, dental flosses and chewing gums for dental cleaning. In particular, the material composition should be effective for combating caries, parodontitis and gingivitis.

### RELATED PRIOR ART

Oral hygiene products are used for the prevention and treatment of diseases of teeth and/or the oral cavity in a multitude of ways. An oral hygiene product can be e.g. a toothpaste, an oral rinse, a spray, a dental floss, a chewing gum, a tongue cleaner or a food supplement. Depending on the application, the oral hygiene product contains several active substances.

Fluoride is employed as active substance against caries in many conventional toothpastes and other oral hygiene products. Fluoride hardens the dental enamel and thus prevents the infiltration of bacteria into the tooth. If one uses an extract of the plant Curcuma Xanthorrhiza, it is possible to combat caries in a totally different way which significantly reduces the number of bacteria causing caries within the oral cavity. U.S. Pat. No. 6,696,404 discloses an antibacterial composition which comprises an extract of Curcuma Xanthorrhiza (CX) and which is used as an oral hygiene product.

The extract of Curcuma Xanthorrhiza (Javanese turmeric) is used as a natural remedy for liver and bilious complaints. The extract of Curcuma Xanthorrhiza (in the following abbreviated as C.X. extract) has an anti-inflammatory and antibacterial effect. In particular, C.X. extract can be used against bacteria like streptococcus mutans which exists within the oral cavity and is responsible for caries. C. X. extract is approved as an article of food. Thus, the intake of this extract is harmless in terms of health even in high concentrations.

Another oral disease is parodontitis which describes an inflammation of the periodontium (jaw bones, gum, dental neck, root cement and periodontal ligament). Dental plaques which contain bacteria are responsible for parodontitis. Another quite similar disease is gingivitis, an inflammation of the gum. U.S. Pat. No. 4,272,512 discloses a composition which comprises folic acid among other substances and which is used for the treatment of gingivitis symptoms like an inflamed gum. In this document of the prior art, the folic acid is used in connection with tranexamic acid in particular.

Folic acid is a vitamin of the vitamin B complex and is also denoted as vitamin B9. Folic acid is inhibitory to irritations and inflammations. Further, folic acid has a positive impact on the immune system and thus promotes a healthy oral mucosa. During pregnancy, a sufficient supply of folic acid is important for preventing the occurrence of foetus malformations (in particular, the neural tube defect).

The present invention aims to provide a material composition with an increased effectiveness for the prevention and treatment of caries, parodontitis and gingivitis.

### SUMMARY OF THE INVENTION

This problem is solved by means of a material composition which comprises both an extract from C.X. and folic acid. The extract from C.X. comprises Xanthorrhizol and further herbal substances. In the framework of the present invention, it is not important by which method the extract from C.X. has been obtained. The usage of an extraction method with carbon dioxide offers advantages in this context, in particular as it does not use any solvent.

Further, it has been noted that the usage of Xanthorrhizol (hydroxyl ar-curcumen) which is part of C.X. instead of an extract from C.X. which comprises Xanthorrhizol and typically also ar-curcumen and beta-curcumen leads to the advantages of the present invention. Xanthorrhizol typically has around 25 weight % of the Xanthorrhiza oil.

When combining these two substances, a surprising synergetic effect is achieved. As a clinical study of the applicant which is further detailed below shows, the combination of an extract from C.X. or Xanthorrhizol and folic acid in a toothpaste leads to a significant reduction of the risk of disease for the three dental diseases caries, parodontitis and gingivitis. In particular, such a toothpaste reduces the risk of disease for all three dental diseases (caries, parodontitis, gingivitis) by 80 to 90 percent. This high effectiveness of the combination can only be explained by the fact that the two substances (the extract from C.X. or Xanthorrhizol and folic acid) mutually increase their effectiveness. This has been unknown so far.

In a preferred embodiment, the concentration of folic acid in the material composition is between 0.02 weight % and 0.2 weight %. The concentration of the extract from C.X. in the material composition is preferably at least 0.005 weight %, in a particularly preferred embodiment at least 0.01 weight % and even more preferred at least 0.03 weight %. If Xanthorrhizol is used instead of the extract from C.X., its concentration in the material composition should preferably be at least 0.0015 weight %, particularly preferred at least 0.003 weight % and even more preferred at least 0.01 weight %.

In a preferred embodiment of the invention, the material composition additionally comprises the vitamins B6 and B12. The addition of the vitamins B6 and B12 further increases the effectiveness of the oral hygiene product with respect to parodontitis and gingivitis.

In a preferred embodiment, the material composition does not contain any fluoride which is in contrast to traditional material compositions for the prevention of caries. The high effectiveness of the material composition of the present invention is the reason why one does not need the addition of fluoride in a preferred embodiment of the invention.

Due to its goods compatibility and high effectiveness, an oral hygiene product according to the present invention can particularly be used during pregnancy as well.

The material composition according to the present invention is suitable for producing an oral hygiene product and in particular can be an ingredient (apart from the corresponding typical ingredients) in a toothpaste, an oral spray, an oral rinse, a dental floss, a chewing gum for dental cleaning or a food supplement.

### DETAILED DESCRIPTION OF THE INVENTION

The applicant has conducted a clinical study during a period of 8 years in which the effect of different toothpastes on the prevalence of the dental diseases caries, parodontitis and gingivitis has been studied, wherein each of the different toothpastes comprises different effective substances or combinations thereof.

There were 9 groups in this clinical study, 3 groups of which consisted of pregnant women. The other 6 groups consisted of half men and half women. Further, the test persons were selected and grouped in such a way that equivalent test groups resulted with respect to the oral health state of the group members at the beginning of the study, the general health of the group members, the age of the group members and with respect to the mental abilities of the group members to conduct an at least average oral hygiene.

The oral hygiene of the participants of the study has been standardised by intensive oral hygiene training. Prior to the beginning of the study, each test person was instructed about professional tooth cleaning and was given the same tools for oral hygiene except for the toothpaste (tooth brush).

All test groups were given the same basic toothpaste. This basic toothpaste of the study consisted of water, humectants, an abrasive, aroma, sweetener (saccharine) and fluoride. The basic toothpaste of only one test group did not contain any fluoride (see group 9 below). The toothpastes of the various test groups differ in that the basic toothpaste was supplemented with additional substances:

Group 1: basic toothpaste only (control group)

Group 2: addition of C.X. extract and folic acid to the basic toothpaste

Group 3: addition of C.X. extract to the basic toothpaste

Group 4: addition of folic acid to the basic toothpaste

Group 5: addition of C.X. extract, folic acid, B6 and B12 to the basic toothpaste

Group 6: addition of B6 and B12 to the basic toothpaste

Group 7: pregnant women with basic toothpaste

Group 8: pregnant women with addition of C.X. extract and folic acid to the basic toothpaste

Group 9: pregnant women with addition of C.X. extract and folic acid to the basic toothpaste without fluoride

Here, the following concentrations of the substances have been added to the corresponding toothpastes:

C.X. extract: 0.03 weight % (the substance was extracted from biologically cultivated plants by means of CO₂ extraction)

Folic acid: 0.08 weight %

Vitamin B6: 0.01 weight % and

Vitamin B12: 0.002 weight %.

It is, however, understood that toothpastes with other concentrations of folic acid, C.X. extract, B6 and/or B12 can also be advantageous under certain circumstances and thus belong to the subject matter of the present invention.

The risk of caries has been quantified by measuring the number of freshly occurring carious teeth in relation to control group 1 or 7 (for pregnant women), whereby the control groups are only using the basic toothpaste. The measurements of freshly occurring carious teeth in groups 1 and 7 were taken as reference measure (100%).

Similarly, the occurring gingivitis and parodontitis of the various test groups was measured in relation to control group 1 or 7 (for pregnant women). The measurements for groups 1 and 7 were again taken as 100%.

All test persons of groups 1 to 6 were examined for evaluation purposes once in each quarter, i.e. four times a year. Groups 7 to 9 of pregnant women were examined once a month. All above-described measurement methods for dental diseases were standardised and not changed during the entire duration of this study. In detail, the result of the study with respect to the three dental diseases is the following:

### RISK OF CARIES

A toothpaste with the addition of C.X. extract alone was able to reduce the risk of caries by 20 to 25% (group 3). In contrast, a toothpaste with folic acid alone had no effect on the risk of caries (group 4). The addition of C.X. extract and folic acid in combination has reduced the risk of caries by up to 80% (groups 2, 8 and 9). Thus, a significant synergetic effect of the combination of C.X. extract and folic acid arises which clearly exceeds the effect of C.X. extract alone.

Besides, it is remarkable that the result for the risk of caries is the same for group 9 of pregnant women who were using the toothpaste without fluoride. This leads to the conclusion that one does not need the addition of fluoride for pregnant women without increasing their risk of caries.

The addition of vitamins B6 and B12 in addition to C.X. extract and folic acid had no effect on the risk of caries (group 5); the addition of vitamins B6 and B12 alone had no effect on the risk of caries, either (group 6).

### RISK OF GINGIVITIS

The toothpaste with addition of C.X. extract alone was able to reduce the risk of gingivitis by 15% (group 3). The toothpaste with folic acid alone was able to reduce the risk of gingivitis by up to 30% (group 4). In contrast, a toothpaste with the combination of C.X. extract and folic acid reduces the risk of gingivitis by up to 82% (group 2). This reduction by 82% is significantly lower than the sum of the single effects 15% and 30%.

For groups 8 and 9 of pregnant women, the combination of C.X. extract and folic acid was measured to reduce the risk of gingivitis by even more than 95%.

The addition of vitamins B6 and B12 in addition to C.X. extract and folic acid was able to reduce the risk of gingivitis for male test persons by up to 91% (male part of group 5), whereas the addition of vitamins had no additional effect for female test persons (female part of group 5). Likewise, a toothpaste with the addition of vitamins B6 and B12 alone had no significant effect on the risk of gingivitis (group 6).

### RISK OF PARODONTITIS

The toothpaste with addition of C.X. extract alone was able to reduce the risk of parodontitis by 18% (group 3), whereas the addition of folic acid alone was able to reduce the risk of parodontitis by up to 35% (group 4). In contrast, a toothpaste with the combination of C.X. extract and folic acid reduced the risk of parodontitis by up to 79% (group 2), which again is significantly more than the sum of 18% and 35%.

For groups 8 and 9 of pregnant women, the toothpaste which comprises the combination of C.X. extract and folic acid even obtained a reduction in the risk of parodontitis by more than 90%.

As in the case of gingivitis, the addition of vitamins B6 and B12 in addition to C.X. extract and folic acid has been able to reduce the risk of parodontitis for male test persons by up to 91% (compared to 79% for a combination of C.X. extract and folic acid) (male part of group 5), whereas again the addition of vitamins B6 and B12 had no additional effect for the female test persons (female part of group 5). Likewise, a toothpaste with the addition of vitamins B6 and B12 had no significant effect on the risk of parodontitis (group 6).

Conclusions: All in all, a toothpaste with a combination of C.X. extract and folic acid has been able to significantly reduce the prevalence for all three dental diseases (between 80 and 90%). This considerable reduction of the risk of disease was surprising, as the two substances C.X. extract and folic acid are not nearly as effective when taken alone and a synergetic effect was not expected. The present study, however, proves a synergetic effect of C.X. extract and folic acid for oral health. Further, a special effect for pregnant women resulting therefrom is that, due to the combination of C.X. extract and folic acid, it is possible to abandon fluoride without reducing the protection against caries. The addition of vitamins B6 and B12 in addition to C.X. extract and folic acid further reduces the risk of disease for gingivitis and parodontitis, but only for male patients.

Even though the combination of C.X. extract and folic acid was used in a toothpaste in the framework of the above-described clinical study, the usage of this combination of substances in an oral rinse, a dental floss, a chewing gum, an oral spray, a tongue cleaner or a food supplement is also planned, as one can expect similar synergetic effects between C.X. extract and folic acid in these applications.

1. A material composition characterized in that it comprises
a. an extract from Curcuma Xanthorrhiza; and
b. the substance folic acid.

2. A material composition characterized in that it comprises
a. Xanthorrhizol; and
b. the substance folic acid.

3. A material composition according to claim 1, characterized in that it additionally comprises the vitamins B6 and B12.

4. A material composition according to claim 1, characterized in that it does not comprise any fluoride.

5. A material composition according to claim 1, characterized in that the concentration of folic acid is between 0.02 weight % and 0.2 weight %.

6. A material composition according to claim 1, characterized in that the concentration of Curcuma Xanthorrhiza is at least 0.005 weight %.

7. A material composition according to claim 2, characterized in that the concentration of Xanthorrhizol is at least 0.0015 weight %.

8. Oral hygiene product, characterized it that it comprises a material composition according to claim 1

9. Toothpaste, characterized in that it comprises a material composition according to claim 1

10. Oral rinse, characterized it that it comprises a material composition according to claim 1

11. Dental floss, characterized in that it comprises a material composition according to claim 1

12. Chewing gum for dental cleaning, characterized it that it comprises a material composition according to claim 1

13. Tongue cleaner, characterized in that it comprises a material composition according to claim 1

14. Food supplement, characterized in that it comprises a material composition according to claim 1

15. Usage of a material composition according to claim 1 for the treatment of caries.

16. Usage of a material composition according to claim 1 for the treatment of gingivitis.

17. Usage of a material composition according to claim 1 for the treatment of parodontitis.

18. Usage of a material composition according to claim 1 during pregnancy.
